(19) United States
(12) Patent Application Publication
(10) Pub. No.: US 2023/0241076 A1
(43) Pub. Date: Aug. 3, 2023

(54) HISTONE-ACETYLATION-MODULATING AGENTS FOR THE TREATMENT AND PREVENTION OF ORGAN INJURY

(71) Applicant: The Regents of the University of Michigan, Ann Arbor, MI (US)

(72) Inventors: Yuqing E. Chen, Ann Arbor, MI (US); Bertram Pitt, Ann Arbor, MI (US); Ienglam Lei, Ann Arbor, MI (US); Shuo Tian, Ann Arbor, MI (US); Francis Pagani, Ann Arbor, MI (US); Paul C. Tang, Ann Arbor, MI (US); Zhong Wang, Ann Arbor, MI (US)

(21) Appl. No.: 18/003,431

(22) PCT Filed: Jun. 29, 2021
(86) PCT No.: PCT/US2021/039650

§ 371 Date: Dec. 27, 2022

Related U.S. Application Data
(60) Provisional application No. 63/045,784, filed on Jun. 29, 2020.

(51) Int. Cl. A61K 31/551; A61K 31/185; A61K 31/192; A61K 31/166; A61K 31/19; A61K 31/455; A61K 31/4745; A61K 31/5517; A61K 31/4439; A61K 31/517; A61K 31/437; A61K 31/5377; A61K 31/5025; A61K 31/585; A61K 31/575; A61K 31/381; A61K 31/12; A61K 31/26; A61K 31/05; A61K 31/353; A61K 31/497; A61K 31/225; A61K 31/36

(52) U.S. Cl. CPC A61K 31/551; A61K 31/185; A61K 31/192; A61K 31/166; A61K 31/19; A61K 31/455; A61K 31/4745; A61K 31/5517; A61K 31/4439; A61K 31/517; A61K 31/437; A61K 31/5377; A61K 31/5025; A61K 31/585; A61K 31/575; A61K 31/381; A61K 31/12; A61K 31/26; A61K 31/05; A61K 31/353; A61K 31/497; A61K 31/225; A61K 31/36

(57) ABSTRACT

Provided herein are agents that modulate histone acetylation and pathways downstream thereof, and methods for the treatment and prevention of organ injury therewith. In particular, provided herein are combinations of histone deacetylase (HDAC) inhibitors, bromodomain and extraterminal-containing protein family (BET) inhibitors, promoters of histone acetyl transferase (HAT) activity, mineralocorticoid receptor (MR) antagonists, nuclear factor erythroid 2-related factor 2 (NRF2) activators, and/or aldehyde dehydrogenase (ALDH) agonists, and methods of use thereof for the treatment and prevention of heart injury.

### CROSS-REFERENCE TO RELATED APPLICATION

The present application claims priority to U.S. Provisional patent application Ser. No. 63/045,784, filed Jun. 29, 2020, which is hereby incorporated by reference in its entirety.

### FIELD

Provided herein are agents that modulate histone acetylation and pathways downstream thereof, and methods for the treatment and prevention of organ injury therewith. In particular, provided herein are combinations of histone deacetylase (HDAC) inhibitors, bromodomain and extraterminal-containing protein family (BET) inhibitors, promoters of histone acetyl transferase (HAT) activity, mineralocorticoid receptor (MR) antagonists, nuclear factor erythroid 2-related factor 2 (NRF2) activators, and/or aldehyde dehydrogenase (ALDH) agonists, and methods of use thereof for the treatment and prevention of heart injury.

### BACKGROUND

Histone acetylation serves as a major post-translational modification (PTM) mark to form the histone acetylation code, which is read by specific epigenetic factors to mediate transcriptional and other cellular responses (Refs. 1-3; incorporated by reference in their entireties) (FIG. 1). The histone acetylation code is maintained and regulated by “writers,” “erasers,” and “readers” (FIG. 1). Histone acetylation has been identified to play numerous roles in organ normal function, pathogenesis and protection/repair, such as pro proliferation, anti-cell death, and anti-inflammation (Refs. 4-5; incorporated by reference in their entireties). The histone acetylation code is disrupted/erased after heart attack or other organ injuries (refs. 6-8; incorporated by reference in their entireties).

### SUMMARY

Provided herein are agents that modulate histone acetylation and pathways downstream thereof, and methods for the treatment and prevention of organ injury therewith. In particular, provided herein are combinations of HDAC inhibitors, BET inhibitors, promoters of histone HAT activity, MR antagonists, NRF2 activators, and/or ALDH agonists, and methods of use thereof for the treatment and prevention of heart injury.

In some embodiments, provided herein are systems comprising a combination of therapeutic/prophylactic agents for administration to a subject for the treatment/prevention of organ/tissue injury, wherein the combination of therapeutic/prophylactic agents comprises two or more (e.g., 3, 4, 5, 6, 7, 8, or more) histone acetylation code agents. In some embodiments, the two or more histone acetylation code agents are selected from a histone deacetylase (HDAC) inhibitor, a bromodomain and extraterminal-containing protein family (BET) inhibitor, a promoter of histone acetyl transferase (HAT) activity, a mineralocorticoid receptor (MR) antagonist, a nuclear factor erythroid 2-related factor 2 (NRF2) activator, and a aldehyde dehydrogenase (ALDH) agonist. In some embodiments, systems comprise an HDAC inhibitor. In some embodiments, the HDAC inhibitor is selected from hydroxamic acid, depsipeptide, benzamide, electrophilic ketone, phenylbutyrate, valproic acid (VPA), a VPA derivative, and nicotinamide. In some embodiments, systems comprise a BET inhibitor. In some embodiments, the BET inhibitor comprises a thienodiazepine moiety or a derivative or variant thereof. In some embodiments, the BET inhibitor is selected from JQ1, I-BET 151, I-BET 762, OTX-015, TEN-010 (JQ2), CPI-203, CPI-0610, olinone, RVX-208, ABBV-744, LY294002, AZD5153, MT-1, and MS645. In some embodiments, systems comprise a MR antagonist. In some embodiments, the MR antagonist is selected from spironolactone, eplerenone, canrenoic acid, canrenone, and drospirenone. In some embodiments, systems comprise a NRF2 activator. In some embodiments, the NRF2 activator is selected from alpha-lipoic acid, curcumin, sulforaphane, resveratrol, polyresveratrol, genistein, andrographolidesiliphos, quercetin, dimethyl fumarate (DMF), oltipraz (4-methyl-5(pyrazinyl-2)-1-2-dithiole-3-thione), and ursodiol (ursodeoxycholic acid). In some embodiments, systems comprise an ALDH agonist. In some embodiments, the ALDH agonist is selected from Alda-1, Alda-89, Alda-52, Alda-59, Alda-72, Alda-71, Alda-53, Alda-54, Alda-61, Alda-60, Alda-66, Alda-65, Alda-64, Alda-84. In some embodiments, systems comprise a promoter of HAT activity. In some embodiments, the promoter of HAT activity is selected from acetyl-CoA and a carbon source precursor to acetyl-CoA. In some embodiments, the carbon source precursor to acetyl-CoA is selected from citrate, acetate, pyruvate, and octanoate. In some embodiments, two or more histone acetylation code agents are combined into a single formulation. In some embodiments, two or more histone acetylation code agents are separately formulated. In some embodiments, two or more histone acetylation code agents are separately formulated but packaged together in a kit. In some embodiments, two or more histone acetylation code agents are separately packaged.

In some embodiments, provided herein are methods of treating/preventing organ/tissue injury in a subject comprising co-administering two or more histone acetylation code agents described herein to the subject. In some embodiments, the subject has suffered a tissue and/or organ injury. In some embodiments, the subject suffers from a disease or condition, or has suffered a physiological event, that causes tissue and/or organ injury. In some embodiments, the subject is at elevated risk of a disease, condition, or physiological event, that causes tissue and/or organ injury. In some embodiments, the tissue and/or organ injury comprises cardiac damage. In some embodiments, the tissue and/or organ injury comprises ischemic damage. In some embodiments, the subject has suffered a myocardial infarction. In some embodiments, the histone acetylation code agents are administered orally or parenterally.

In some embodiments, provided herein are methods of treating/preventing organ damage comprising (a) intravenously administering in clinical setting a first combination of histone acetylation code agents to a subject that has suffered and ischemic event; and (b) orally administering a second combination of histone acetylation code agents to the subject. In some embodiments, the first combination and the second combination comprise the same histone acetylation code agents, but are formulated for intravenous and oral administration, respectively. In some embodiments, the first combination and the second combination comprise different histone acetylation code agents. In some embodiments, oral administration is in a clinical setting and/or an out-patient setting. In some embodiments, oral administration continues for at least 1 week (e.g., 2 weeks, 3 weeks, 4 weeks, 5 weeks, 6 weeks, 8 weeks, 10 week, or more) after the intravenous administration.

### BRIEF DESCRIPTION OF THE DRAWINGS

FIG. 1. Schematic depicting the roles of writers, erasers, and readers in the histone acetylation code.

FIG. 2. Canrenone (50 μM) improves murine donor heart function after 16 h of cold storage followed by ex vivo perfusion. max dp/dt (contraction) and min dp/dt (relaxation) were calculated by average the max/min dp/dt over a 20 minutes period after different cold storage periods followed by 60 minutes of reperfusion. *P<0.05, **P<0.01. N=8 at each time point.

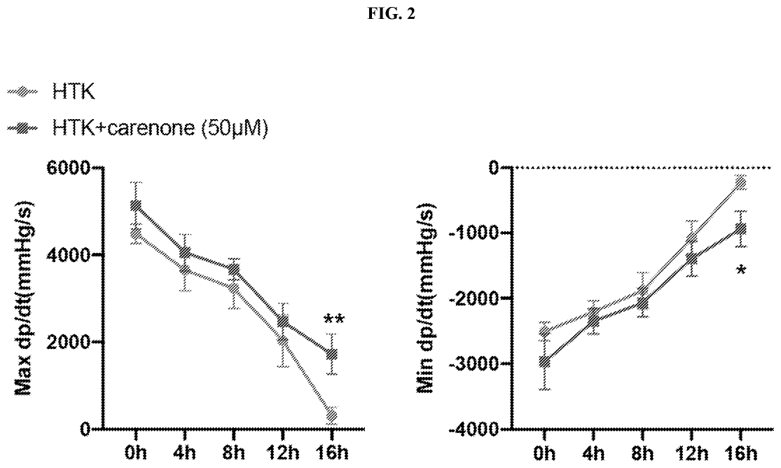

FIG. 3. Cardiomyocyte specific deletion of mineralocorticoid receptor improve murine donor heart function after 16 h of cold storage followed by ex vivo perfusion. WT and Myh6CreErt2; MRf/f mice was treated with tamoxifen at dose of 80 mg/kg/day for 5 days. Donor heart was isolated and store for 16 hr, then subjected to perfusion. max dp/dt (contraction) and min dp/dt (relaxation) were calculated by average the max/min dp/dt over a 20 minutes period after different cold storage periods followed by 60 minutes of reperfusion. *P<0.05, n=8.

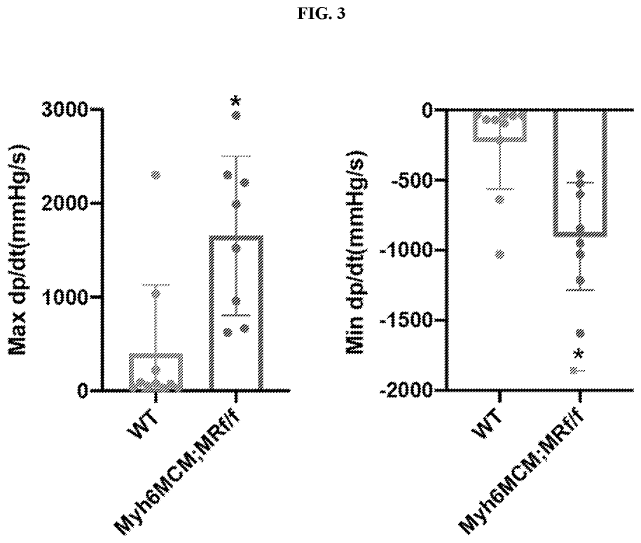

FIG. 4. Canrenone (50 μM) improve murine donor heart function after 16 h of cold storage followed by 24 h of heterotopic transplantation. Donor heart was isolated and store with or without canrenone for 16 hr, then the donor hearts were heterotopic transplanted to receipt cervical region. max dp/dt (contraction) and min dp/dt (relaxation) were calculated by average the max/min dp/dt for 1 minute period after 24 hr of transplantation using Millar Catheter. *P<0.05, n=8.

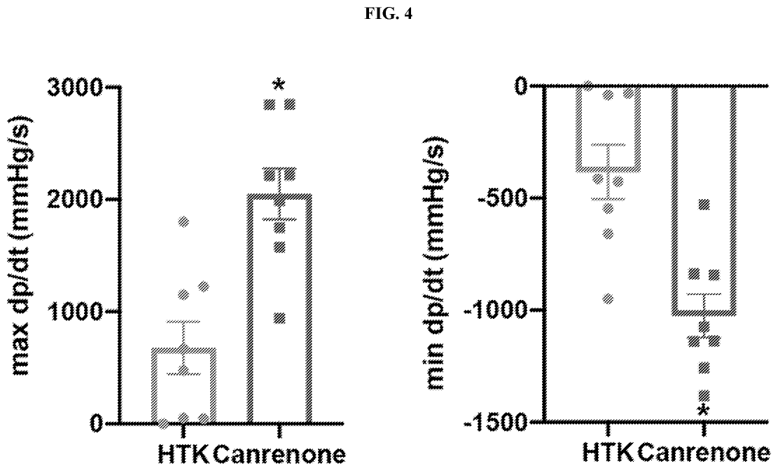

FIG. 5. Canrenone (50 μM) improve pig donor heart function after 10 h of cold storage followed by ex vivo prefusion. Pig hearts were perfused with 1 L HTK solution after cross clamp. Addition 2 L of HTK with or without canrenone were perfused and stored for 10 hr on ice. Pig hearts were perfused in ex vivo system with blood and Krebs buffer (1:4, hemoglobin concentration: 5.2-5.6 g/L). max dp/dt (contraction) and min dp/dt (relaxation) were calculated by average the max/min dp/dt for 10 minute period after 30 minutes of reperfusion using Millar Catheter. *P<0.05, **P<0.01. N=4.

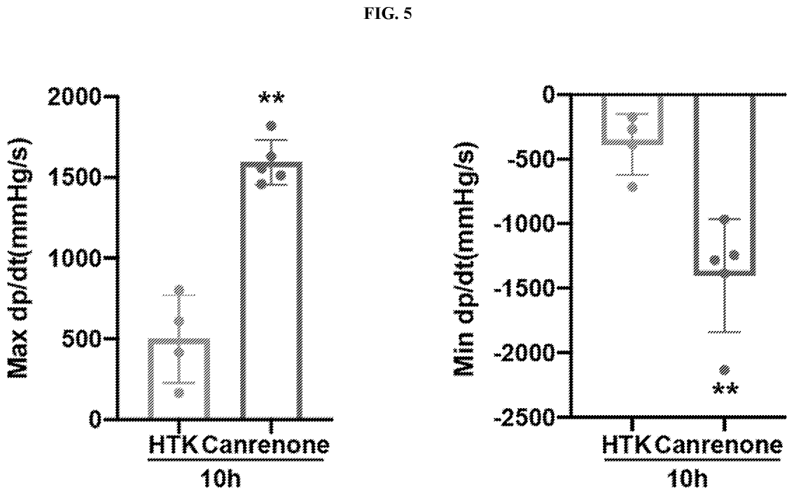

FIGS. 6A-D. Cold storage of donor heart induces liquid-liquid phase separation of MR. FIG. 6A. Purified GFP or GFP-MR protein in 10% PEG solution. FIG. 6B. Turbidity of purified GFP and GPF-MR protein 10% PEG solution. FIG. 6C. Droplet experiment showing that GFP-MR can form condensate in vitro. FIG. 6D. Cold storage of donor heart upregulates MR expression as well as induces nuclear condensates formation.

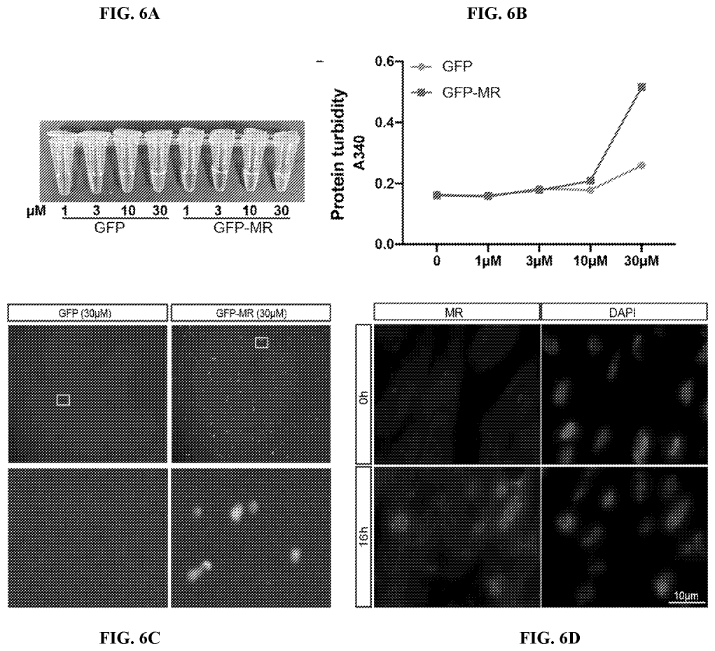

FIG. 7, panels A-C. VPA improve donor heart ischemic tolerance in part through upregulated of Irg1. Panel A. Cold storage of donor heart induce Irg1 expression, VPA treatment further stimulates Irg1 upregulation. Panels B and C. Irg1 deletion reduce the protective effect of VPA in donor heart function after 16 hr of cold storage followed by reperfusion.

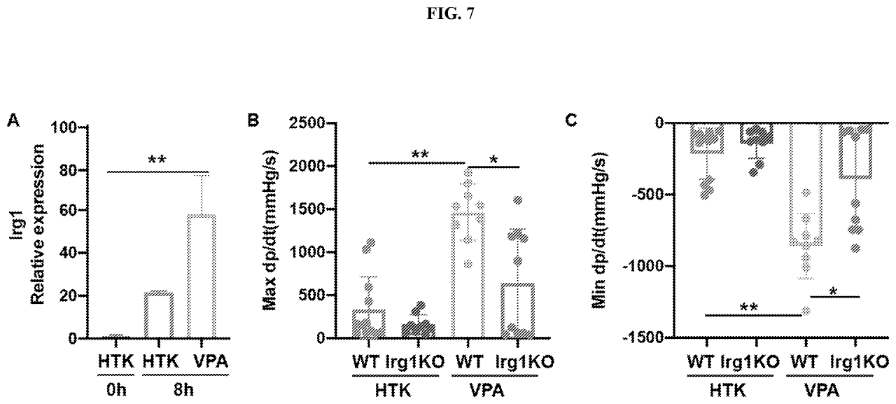

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “an agent” is a reference to one or more agents and equivalents thereof known to those skilled in the art, and so forth.

As used herein, the term “about,” when referring to a value is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “histone acetylation code” refers to posttranslational modifications (e.g., acetylation) to histone proteins that serve as epigenetic markers and modify the expression of various genes and the activities of downstream pathways.

As used herein, the terms “histone acetylation code agent,” “histone acetylation code drug,” of variations thereof refer to agents or drugs (e.g., small molecules, peptide, antibodies, nucleic acids, etc.) that modify or alter (e.g., increase or decrease) the activity or expression of one or more factors involved in writing, erasing, or reading the histone acetylation code of are involved in pathways upstream or downstream thereof.

As used herein, the term “system” refers to two or more elements that are present together (e.g., as in a kit) but are not necessarily formulated into a single composition or contained in the same packaging.

The term “effective dose” or “effective amount” refers to an amount of an agent which results in a desired biological outcome (e.g., inhibition of osteoclast production and/or activity).

As used herein, the terms “administration” and “administering” refer to the act of providing a therapeutic, prophylactic, or other agent to a subject for the treatment or prevention of one or more diseases or conditions. Exemplary routes of administration to the human body are through space under the arachnoid membrane of the brain or spinal cord (intrathecal), the eyes (ophthalmic), mouth (oral), skin (topical or transdermal), nose (nasal), lungs (inhalant), oral mucosa (buccal), ear, rectal, vaginal, by injection (e.g., intravenously, subcutaneously, intratumorally, intraperitoneally, etc.) and the like.

As used herein, the term “treat,” and linguistic variations thereof, encompasses therapeutic measures, while the term “prevent” and linguistic variations thereof, encompasses prophylactic measures, unless otherwise indicated (e.g., explicitly or by context).

As used herein, the terms “co-administration” and “co-administering” refer to the administration of at least two agent(s) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

As used herein, the term “pharmaceutical composition” refers to the combination of an active agent with a carrier, inert or active, making the composition especially suitable for diagnostic or therapeutic use in vitro, in vivo or ex vivo.

The term “pharmaceutically acceptable” as used herein, refers to compositions that do not substantially produce adverse reactions, e.g., toxic, allergic, or immunological reactions, when administered to a subject.

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), and various types of wetting agents, any and all solvents, dispersion media, coatings, sodium lauryl sulfate, isotonic and absorption delaying agents, disintigrants (e.g., potato starch or sodium starch glycolate), and the like. The compositions also can include stabilizers and preservatives. For examples of carriers, stabilizers and adjuvants, see, e.g., Martin, Remington's Pharmaceutical Sciences, 15th Ed., Mack Publ. Co., Easton, Pa. (1975), incorporated herein by reference in its entirety.

### DETAILED DESCRIPTION

Provided herein are agents that modulate histone acetylation and pathways downstream thereof, and methods for the treatment and prevention of organ injury therewith. In particular, provided herein are combinations of HDAC inhibitors, BET inhibitors, promoters of histone HAT activity, MR antagonists, NRF2 activators, and/or ALDH agonists, and methods of use thereof for the treatment and prevention of heart injury.

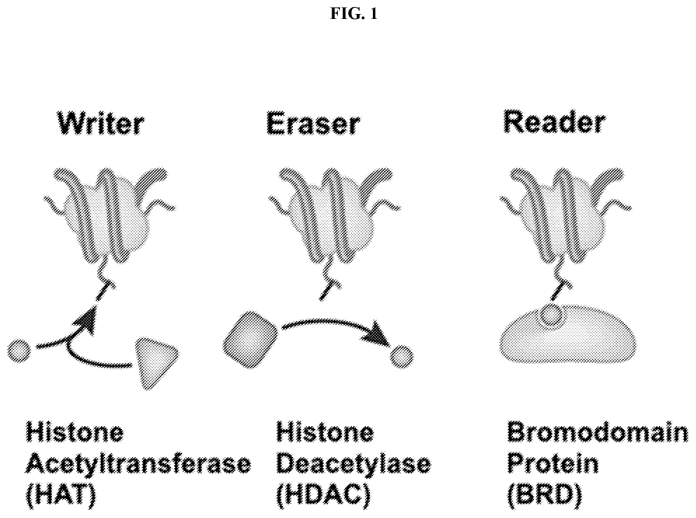

Histone acetylation is a post-translational modification (PTM) of histone proteins, which is regulated by specific proteins and read by specific epigenetic factors to mediate transcription and other cellular responses (Refs. 1-3; incorporated by reference in their entireties). The histone acetylation code is maintained and regulated by its writers, erasers, and readers (FIG. 1). Histone acetylation plays numerous roles in organ function, pathogenesis and protection/repair, such as (Refs. 4, 5; incorporated by reference in their entireties). The code is disrupted/erased after organ injury (e.g., heart attack) (Refs 6-8: incorporated by reference in their entireties). Therapeutic restoration of histone acetlyation, and/or prophylactic protection prior to disruption, by targeting different components of the histone acetylation code with a combination of drugs provides an effective strategy to prevent cell damage and promote organ repair after injury.

Histone acetyl transferases (HATs) transfer acetyl groups to histone proteins. Acetyl-CoA is an evolutionarily conserved intermediate energy metabolite in TCA cycle for ATP production, as well as being the substrate for histone acetyltransferases (HATs) to transfer the acetyl-group to histone residues for histone acetylation (Refs. 9-10; incorporated by reference in their entireties). Acetyl-CoA serves as a gatekeeper or sensor between energy metabolism and gene expression/chromatin epigenetics by modulating the chromatin landscape in response to key nutritional cues (Refs. 11-18; incorporated by reference in their entireties). Metabolic carbon sources that swiftly produce acetyl-CoA (e.g., acetate, pyruvate, octanoate (8C), etc.) improve heart function in ischemia reperfusion (IR) rats. In particular, 8C administration of 160 mg/kg results in significant heart functional recovery when i.p. injected once at the time of reperfusion (Ref 19; incorporated by reference in its entirety).

Histone deacetylases (HDACs), which remove acetyl-group from histones to erase histone acetylation code, have been studied extensively for drug discovery. Numerous HDAC inhibitors have been identified in treating disease. For example, valproic acid (VPA), an FDA approved histone deacetylases (HDAC) inhibitor drug for bipolar disorders, protects the heart against AMI injury in rats (Refs. 12-13; incorporated by reference in their entireties). VPA has been shown in large epidemiologic studies to reduce the incidence of stroke and AMI (Refs. 14-15; incorporated by reference in their entireties). VPA has also been shown to affect the acetylation of the mineralocorticoid receptor (MR) and to reduce fibrosis in the heart, kidneys, lung and liver (Ref 14; incorporated by reference in its entirety).

While the histone acetylation code is typically written by HATs and erased by HDACs, a number of epigenetic factor families, in particular, bromodomain (BRD) containing epigenetic factors, such as BET BRD proteins, recognize the acetylation code and bind to the acetylated histones (Ref 11; incorporated by reference in its entirety). Studies have shown that JQ-1 can effectively halt heart failure and may protect cardiomyocytes under IR conditions (Ref 17; incorporated by reference in its entirety).

Other proteins are also acetylated by histone code writers and erasers in cell protection and organ repair. Histone code writers and erasers often acetylate or deacetylate other proteins to regulate their activities, which is an extension of the histone code. HATs and HDAC inhibitors can acetylate both nuclear factor erythroid 2-related factor 2 (NRF2) and mineralocorticoid receptor (MR) (refs. 14, 16; incorporated by reference in their entireties). The acetylation of NRF2 promotes its activity for cell protection whereas acetylation of MR inhibits its activity for cell protection. Therefore, in some embodiments, NRF2 activators, activators of ALDH, and MR inhibitors find use in histone acetylation code drug combinations.

Provided herein are combinations of histone acetylation code drugs and method of use thereof to treat and/or prevent organ (e.g., heart) damage therewith. In some embodiments, the combinations herein provide synergistic effects on myocardial cell protection, recovery from acute myocardial injury and subsequent thrombosis. The drug combinations herein synergistically in cell protection and organ repair and provide a therapeutic/prophylactic effect that exceeds those of the individual components.

In some embodiments, a subject that has suffered and event or is suffering from a disease or condition that may result in organ (e.g., heart tissue) damage is administered a composition or drug combination herein to treat or prevent damage to the organ. For example, patients suffer organ damage as a result of myocardial infarction. In some embodiments, a subject that suffers a myocardial infarction is administered (e.g., intravenously) a composition or drug combination herein administered a cocktail of histone acetylation drugs at the time of hospitalization after ischemic organ injury and during patients' stay at care unit. Patients will continuously take these drugs orally for a month after they are discharged from the hospital. The goal is to protect cell and organ damage post ischemic organ injury, such as heart, liver, kidney, limb and brain.

In some embodiments, provided herein are combinations of prophylactic and/or therapeutic agents (e.g., for co-administration to a subject) for the treatment and/or prevention of organ injury (e.g., heart injury).

In some embodiments, provided herein are compositions and/or combinations of drugs for administration to a subject comprising a histone deacetylase inhibitor (HDAC inhibitor, HDACi, HDI). HDIs have a long history of use in psychiatry and neurology as mood stabilizers and anti-epileptics. More recently they are being investigated as possible treatments for cancers, parasitic, and inflammatory diseases. Based on the homology of accessory domains to yeast histone deacetylases, the 18 currently known human histone deacetylases are classified into four groups (I-IV): Class I, which includes HDAC1, -2, -3 and -8 are related to yeast RPD3 gene; Class IIA, which includes HDAC4, -5, -7 and -9; Class IIB -6, and -10 are related to yeast Hda1 gene; Class III, also known as the sirtuins are related to the Sir2 gene and include SIRT1-7; Class IV, which contains only HDAC11 has features of both Class I and II. In some embodiments, a histone deacetylase inhibitor that finds use herein is selective for one or more classes of HDAC (e.g., Class I, Class IIA, Class III, and/or Class IV). In some embodiments, a histone deacetylase inhibitor that finds use herein is a general HDAC inhibitor. The “classical” HDIs act exclusively on Class I, II and Class IV HDACs by binding to the zinc-containing catalytic domain of the HDACs. These classical HDIs are classified into several groupings named according to the chemical moiety that binds to the zinc ion (except cyclic tetrapeptides which bind to the zinc ion with a thiol group). Some examples in decreasing order of the typical zinc binding affinity include: hydroxamic acids (or hydroxamates), such as trichostatin A; cyclic tetrapeptides (such as trapoxin B), and the depsipeptides; benzamides; electrophilic ketones; and aliphatic acid compounds such as phenylbutyrate and valproic acid. “Second-generation” HDIs include the hydroxamic acids vorinostat (SAHA), belinostat (PXD101), LAQ824, and panobinostat (LBH589); and the benzamides entinostat (MS-275), tacedinaline (CI994), and mocetinostat (MGCD0103). The sirtuin Class III HDACs are dependent on NAD+ and are, therefore, inhibited by nicotinamide, as well as derivatives of NAD, dihydrocoumarin, naphthopyranone, and 2-hydroxynaphthaldehydes. Any of the above HDIs may find use in embodiments herein.

Valproic acid (VPA) is a histone deacetylase inhibitor with potent anti-inflammatory effects. Inhibition of HDAC leads to epigenetic modifications that cause relaxation of the nucleosome structure allowing the transcription of many gene. VPA attenuate injury in various organ systems such as the lungs, kidneys, and brain. A variety of valproic acid derivatives are well known in the field. Experiments have demonstrated that valproic acid derivatives, such as valnoctamide (VCD) and c-Butyl-propyl-acetamide (SPD), exhibit similar biological characteristics to VPA (Neuman et al. Clinical Biochemistry 46 (2013) 1532-1537; incorporated by reference in its entirety). In some embodiments herein, a composition or drug combination comprises VPA or a valproic acid derivative. Exemplary VPA derivatives include VCD, SPD, and the VPA derivatives depicted in Table 3.

TABLE 1
Exemplary Valproic Acid Derivatives.

| | |
|---|---|
| 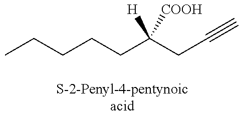 | I |
| 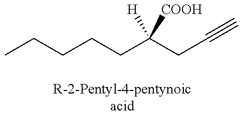 | II |
| 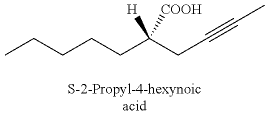 | III |
| 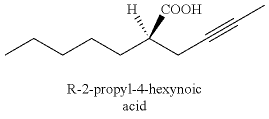 | IV |
| 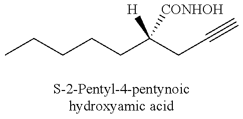 | V |
| 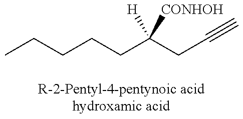 | VI |
| 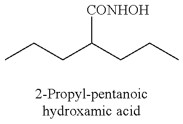 | VII |
| 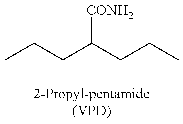 | |
| 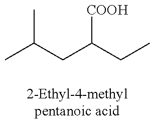 | VIII |
| 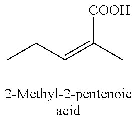 | IX |
| 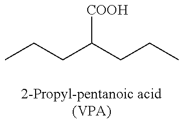 | |

In some embodiments, a suitable VPA derivative comprises a 6-8 member alkyl chain. In some embodiments, the alkyl chain comprises one or more double or triple bonds. In some embodiments, the alkyl chain comprises (CH₂)₆, (CH₂)₇, or CH₂)₈. In some embodiments, the VPA derivative comprises a substituent at the 4 or 5 position of the alkyl chain. In some embodiments, the alkyl chain comprises a substituent at one or more additional positions.

In some embodiments, a valproic acid derivatives are branched carboxylic acids as described by Formula 1:

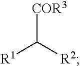

wherein R¹ and R² independently are saturated or unsaturated aliphatic C₂₋₅, which optionally comprises one or several heteroatoms and which may be substituted, R³ is hydroxyl, halogen, alkoxy or an optionally alkylated amino group. Different R¹ and R² residues give rise to chiral compounds. The present invention encompasses the racemic mixtures of the respective compounds. The hydrocarbon chains R¹ and R² may comprise one or several heteroatoms (e.g. O, N, S) replacing carbon atoms in the hydrocarbon chain. Structures very similar to that of carbon groups may be adopted by heteroatom groups when the heteroatoms have the same type of hybridization as a corresponding carbon group. R¹ and R² may be substituted. Possible substituents include hydroxyl, amino, carboxylic and alkoxy groups as well as aryl and heterocyclic groups. In some embodiments, “COR³” is a carboxylic group. In other embodiments, R³ is a halide (e.g., chloride, bromide, etc.), ester, alkoxy, etc. According to the present invention, pharmaceutically acceptable salts of a compound of formula I can be used. Other VPA derivatives understood in the field are also within the scope herein.

In some embodiments, provided herein are compositions and/or combinations of drugs for administration to a subject comprising a bromodomain and extraterminal-containing protein family (BET) inhibitor (BETI). The bromodomain and extraterminal (BET) protein family (BRD2, BRD3, BRD4, and BRDT) are epigenetic readers that, via their bromodomains, regulate gene transcription by binding to acetylated lysine residues on histones and master transcriptional factors (Morgado-Pascual et al. Front. Pharmacol. November 2019, Vol. 10, Article 1315; incorporated by reference in its entirety). In some embodiments, a BET inhibitor comprises a thienodiazepine moiety or a derivative or variant thereof. Examples of BET inhibitors that find use in certain embodiments herein include JQ1, I-BET 151, I-BET 762, OTX-015, TEN-010 (JQ2), CPI-203, CPI-0610, olinone, RVX-208, ABBV-744, LY294002, AZD5153, MT-1, MS645, etc. In some embodiments, a BET inhibitor targets/binds to the first bromodomain (BD1) of a BET protein (e.g., olinone, etc.). In some embodiments, a BET inhibitor targets/binds to the second bromodomain (BD2) of a BET protein (e.g., RVX-208, ABBV-744, etc.). In some embodiments, a BET inhibitor is capable of targeting/binding both bromodomains (BD1/BD2) of a BET protein (e.g., I-BET 151, I-BET 762, OTX-015, TEN-010 (JQ2), CPI-203, CPI-0610, etc.). In some embodiments, a BET inhibitor is bivalent (e.g., AZD5153, MT-1, MS645, etc.).

In some embodiments, provided herein are compositions and/or combinations of drugs for administration to a subject comprising a promoter of histone acetyltransferase (HAT) activity. In some embodiments, a promoter of HAT activity is a acetyl-CoA, the substrate for histone acetyltransferases (HATs) to transfer the acetyl-group to histone residues for histone acetylation (Refs. 9-10; incorporated by reference in their entireties) In some embodiments, a promoter of HAT activity is a carbon source precursor to acetyl-CoA, such as citrate, acetate, pyruvate, and octanoate (8C). In some embodiments, the increased carbon/precursor content results in increase acetylation by HATs of histones.

In some embodiments, provided herein compositions and/or combinations of drugs for administration to a subject comprising a mineralocorticoid receptor antagonist. Mineralocorticoid receptor antagonists bind to and block the activation of mineralocorticoid receptors by mineralocorticoids such as aldosterone. Examples of mineralocorticoid receptor antagonists that find use in embodiments herein include spironolactone (an aldosterone receptor antagonist used for the treatment of hypertension, hyperaldosteronism, edema due to various conditions, hirsutism (off-label) and hypokalemia), eplerenone (an aldosterone receptor antagonist used to improve survival of patients with symptomatic heart failure and to reduce blood pressure), canrenoic acid (metabolized to canrenone in the body), canrenone, and drospirenone (a progestin used in oral contraceptive pills for the prevention of pregnancy and other conditions).

In some embodiments, provided herein compositions and/or combinations of drugs for administration to a subject comprising an aldehyde dehydrogenase agonist. Aldehyde dehydrogenases (ALDHs) are a family of detoxifying enzymes. The human genome has 19 known ALDH genes, but one ALDH, ALDH2, emerges as a particularly important enzyme in a variety of human pathologies. Aldehyde dehydrogenase activators (Aldas) are a family of small molecule activators of ALDHs, exemplified by lda-1 [N-(1,3-benzodioxol-5-ylmethyl)-2,6-dichlorobenzamide, MW 324]. Alda-1 is an allosteric agonist of ALDH2, and corrects a structural defect in the ALDH2*2 mutant present in 8% of the human population. Alda-89 [5-(2-propenyl)-1,3-benzodioxole, commonly known as safrole, MW=162] is a selective activator of acetaldehyde metabolism by ALDH3A1. Other Aldas that find use in embodiments herein include Alda-52 (1-methoxy-naphthalene-2-carboxylic acid (benzo[1,3]dioxol-5-ylmethyl)-amide), Alda-59 (4-[(benzo[1,3]dioxol-5-ylmethyl)-sulfamoyl]-thiophene-2-carboxylic acid amide), Alda-72 (N-benzo[1,3]dioxol-5-ylmethyl-2-(1-oxo-1,2-dihydro-2,3,9-triaza-fluoren-9-yl)-acetamide), Alda-71 (N-(4-methyl-benzyl)-2-(1-oxo-1,2-dihydro-2,3,9-triaza-fluoren-9-yl)-acetamide), Alda-53 (N-benzo[1,3]dioxol-5-ylmethyl-2-chloro-5-[1,2,4]triazol-4-yl-benzamide), Alda-54 (1-ethyl-4-oxo-1,4-dihydro-chromeno[3,4-d]imidazole-8-sulfonic acid (benzo[1,3]dioxol-5-ylmethyl)-amide), Alda-61 (5-(3,4-dimethyl-isoxazol-5-yl)-2-methyl-N-(4-methyl-benzyl)-benzene sulfonamide), Alda-60 (2-methyl-N-(4-methyl-benzyl)-5-(3-methyl-isoxazol-5-yl)-benzene sulfonamide), Compound Alda-66 (2-(2-isopropyl-3-oxo-2,3-dihydro-imidazo[1,2-c]quinazolin ylsulfanyl)-N-(4-methyl-benzyl)-propionamide), Alda-65 (N-(3,5-dimethyl-phenyl)-2-(2-isopropyl-3-oxo-2,3-dihydro-imidazo[1,2-c]quinazolin-5-ylsulfanyl)-acetamide), Alda-64 (2-(azepane-1-carbonyl)-N-(2-chloro-benzyl)-2,3-dihydro-benzo[1,4]dioxine-6-sulfonamide), Alda-84 (N-(2-hydroxy-phenyl)-3-phenyl-acrylamide), and any other suitable Aldas known in the field, for example, those described in U.S. Pub. No. 2010/0113423; incorporated by reference in its entirety.

In some embodiments, provided herein are compositions and/or combinations of drugs for administration to a subject comprising an activator of nuclear factor erythroid 2-related factor 2 (NRF2). In some embodiments, a NRF2 activator is selected from alpha-lipoic acid, curcumin, sulforaphane, resveratrol, polyresveratrol, genistein, andrographolidesiliphos, quercetin, dimethyl fumarate (DMF), oltipraz (4-methyl-5(pyrazinyl-2)-1-2-dithiole-3-thione), and ursodiol (ursodeoxycholic acid).

In some embodiments, provided herein is combination therapeutic and/or prophylactic comprising two or more (e.g., 2, 3, 4, 5) of a BET inhibitor, promoter of HAT activity, MR antagonist, NRF2 activators, ALDH agonist, NRF2 activator, and/or HDAC inhibitor. In some embodiments, a combination therapeutic and/or prophylactic comprises a BET inhibitor herein and one or more or a promoter of HAT activity, an MR antagonist, aNRF2 activator, an ALDH agonist, a NRF2 activator, and/or a HDAC inhibitor. In some embodiments, a combination therapeutic and/or prophylactic comprises a promoter of HAT activity herein and one or more or a BET inhibitor, an MR antagonist, aNRF2 activator, an ALDH agonist, a NRF2 activator, and/or a HDAC inhibitor. In some embodiments, a combination therapeutic and/or prophylactic comprises an MR antagonist herein and one or more or a BET inhibitor, a promoter of HAT activity, aNRF2 activator, an ALDH agonist, a NRF2 activator, and/or a HDAC inhibitor. In some embodiments, a combination therapeutic and/or prophylactic comprises a NRF2 activator herein and one or more or a BET inhibitor, a promoter of HAT activity, an MR antagonist, an ALDH agonist, and/or a HDAC inhibitor. In some embodiments, a combination therapeutic and/or prophylactic comprises an ALDH agonist herein and one or more or a BET inhibitor, a promoter of HAT activity, an MR antagonist, a NRF2 activator, a NRF2 activator, and/or a HDAC inhibitor. In some embodiments, a combination therapeutic and/or prophylactic comprises a HDAC inhibitor herein and one or more or a BET inhibitor, a promoter of HAT activity, an MR antagonist, a NRF2 activator, a NRF2 activator, and/or an ALDH agonist.

A drug combination may comprise a single agent from any of the categories of histone acetylation code agents herein (e.g., BET inhibitors, promoters of HAT activity, MR antagonists, NRF2 activators, ALDH agonists, NRF2 activators, HDAC inhibitors, etc.). In some embodiments, a drug combination comprises a two or more agents from a category of histone acetylation code agents herein (e.g., BET inhibitors, promoters of HAT activity, MR antagonists, NRF2 activators, ALDH agonists, NRF2 activators, HDAC inhibitors, etc.).

In some embodiments, the histone acetylation code agents and combinations thereof are administered to a subject to treat or prevent organ damage/injury. In some embodiments, the histone acetylation code agents and combinations thereof are administered to a subject to prevent organ damage/injury from a disease or condition that the subject suffers from or an acute event that the subject has suffered. In some embodiments, the histone acetylation code agents and combinations thereof are administered to a subject to prevent organ damage/injury from a disease, condition, or event that the subject is at elevated risk of (e.g., due to risk factors (e.g., age, genetics, gender, environmental factors, lifestyle, underling health conditions, etc.). In some embodiments, the histone acetylation code agents and combinations thereof are administered to a subject to treat organ damage/injury that has occurred as a result of a disease or condition that the subject suffers from or an acute event that the subject has suffered.

The agent/combination may be administered therapeutically/prophylactically for the treatment/prevention of injury/damage to any tissue, organ, system, etc. and as a result of any suitable condition, disease, or event. For example, the subject may suffer from (or have suffered) or be at risk of disorder/event of the cardiovascular system, the digestive system, the endocrine system, the excretory system, the lymphatic system, the integumentary system, the muscular system, the nervous system, the reproductive system, the respiratory system, and/or the skeletal system. In some embodiments, the disorder/event may be of the heart, salivary glands, esophagus, stomach, liver, gallbladder, pancreas, intestines, colon, rectum, anus, endocrine glands (such as the hypothalamus, pituitary gland, pineal body, thyroid, parathyroid, adrenal glands), kidneys, ureters, bladder, urethra, lymph nodes, tonsils, adenoids, thymus, spleen, skin, muscles, brain, spinal cord, ovaries, fallopian tubes, uterus, vulva, vagina, testes, vas deferens, seminal vesicles, prostate, penis, pharynx, larynx, trachea, bronchi, lungs, diaphragm, cartilage, one or more ligaments, one or more nerves, or one or more tendons of the subject. In particular embodiments, the disorder/event is of the cardiovascular system and the drug combination is administered to treat or prevent myocardial damage. However, the drug combinations herein may be administered in response to a kidney disorder/event (e.g. chronic kidney disease, polycystic kidney disease, etc.), a liver disorder/event (e.g. inherited metabolic defects, chronic viral hepatitis, liver cirrhosis, primary or metastatic liver cancer, etc.), cancer, etc.

Exemplary cardiovascular/cardiac disorders/events for which the combinations herein are administered are selected from the list of aortic dissection, cardiac arrhythmia (e.g. atrial cardiac arrhythmia (e.g. premature atrial contractions, wandering atrial pacemaker, multifocal atrial tachycardia, atrial flutter, atrial fibrillation, etc.), junctional arrhythmias (e.g. supraventricular tachycardia, AV nodal reentrant tachycardia, paroxysmal supra-ventricular tachycardia, junctional rhythm, junctional tachycardia, premature junctional complex, etc.), atrio-ventricular arrhythmias, ventricular arrhythmias (e.g. premature ventricular contractions, accelerated idioventricular rhythm, monomorphic ventricular tachycardia, polymorphic ventricular tachycardia, ventricular fibrillation, etc.), congenital heart disease, myocardial infarction, dilated cardiomyopathy, hypertrophic cardiomyopathy, aortic regurgitation, aortic stenosis, mitral regurgitation, mitral stenosis, Ellis-van Creveld syndrome, familial hypertrophic cardiomyopathy, Holt-Orams Syndrome, Marfan Syndrome, Ward-Romano Syndrome, and/or similar diseases and conditions.

In some embodiments, routes of administration, formation of the desired agents (e.g., a combination of histone acetylation code drugs (e.g., a BET inhibitor, promoter of HAT activity, MR antagonist, NRF2 activators, ALDH agonist, and/or HDAC inhibitor, etc.)), and the pharmaceutical composition are selected to provide efficient and effective delivery. In some embodiments, the therapeutic agents herein (e.g., a combination of histone acetylation code drugs (e.g., a BET inhibitor, promoter of HAT activity, MR antagonist, ALDH agonist, NRF2 activator, and/or HDAC inhibitor, etc.)) are provided in pharmaceutical formulations for administration to a subject by a suitable route. The pharmaceutical formulations described herein can be administered to a subject by multiple administration routes, including but not limited to, oral, parenteral (e.g., intravenous, subcutaneous, intramuscular), intranasal, buccal, topical, rectal, or transdermal administration routes. Moreover, the pharmaceutical compositions described herein (e.g., a combination of histone acetylation code drugs (e.g., a BET inhibitor, promoter of HAT activity, MR antagonist, ALDH agonist, NRF2 activator, and/or HDAC inhibitor, etc.)) are formulated into any suitable dosage form, including but not limited to, aqueous oral dispersions, liquids, gels, syrups, elixirs, slurries, suspensions, aerosols, fast melt formulations, effervescent formulations, lyophilized formulations, tablets, powders, pills, dragees, and capsules.

One may administer the agents and/or compositions in a local rather than systemic manner, for example, via injection of the compound directly into an organ or tissue, often in a depot preparation or sustained release formulation. Such long acting formulations may be administered by implantation (for example subcutaneously or intramuscularly) or by intramuscular injection. Furthermore, one may administer the drug(s) in a targeted drug delivery system, for example, in a liposome coated with organ-specific antibody. The liposomes will be targeted to and taken up selectively by the organ. In addition, the drug(s) may be provided in the form of a rapid release formulation, in the form of an extended release formulation, or in the form of an intermediate release formulation.

Pharmaceutical preparations for oral use can be obtained by mixing one or more solid excipients with the therapeutic agent(s) (e.g., one or a combination of histone acetylation code drugs (e.g., a BET inhibitor, promoter of HAT activity, MR antagonist, ALDH agonist, NRF2 activator, and/or HDAC inhibitor, etc.)) with any suitable substituents and functional groups disclosed herein, optionally grinding the resulting mixture, and processing the mixture of granules, after adding suitable auxiliaries, if desired, to obtain tablets, pills, or capsules. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents may be added, such as the cross-linked croscarmellose sodium, polyvinylpyrrolidone, agar, or alginic acid or a salt thereof such as sodium alginate.

In some embodiments, agents are delivered by inhalation. For administration by inhalation, the agents of combinations of agents described herein (e.g., a combination of histone acetylation code drugs (e.g., a BET inhibitor, promoter of HAT activity, MR antagonist, ALDH agonist, NRF2 activator, and/or HDAC inhibitor, etc.)) may be in a form as an aerosol, a mist or a powder. In some embodiments, pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Buccal formulations that include the agents or combinations described herein (e.g., a combination of histone acetylation code drugs (e.g., a BET inhibitor, promoter of HAT activity, MR antagonist, ALDH agonist, NRF2 activator, and/or HDAC inhibitor, etc.)) may be administered using a variety of formulations which include, but are not limited to, U.S. Pat. Nos. 4,229,447, 4,596,795, 4,755,386, and 5,739,136.

In some embodiments, the agents or combinations described herein (e.g., a combination of histone acetylation code drugs (e.g., a BET inhibitor, promoter of HAT activity, MR antagonist, ALDH agonist, NRF2 activator, and/or HDAC inhibitor, etc.)) are delivered transdermally. Transdermal formulations described herein may be administered using a variety of devices including but not limited to, U.S. Pat. Nos. 3,598,122, 3,598,123, 3,710,795, 3,731,683, 3,742,951, 3,814,097, 3,921,636, 3,972,995, 3,993,072, 3,993,073, 3,996,934, 4,031,894, 4,060,084, 4,069,307, 4,077,407, 4,201,211, 4,230,105, 4,292,299, 4,292,303, 5,336,168, 5,665,378, 5,837,280, 5,869,090, 6,923,983, 6,929,801 and 6,946,144; incorporated by reference in their entireties.

In some embodiments, the agents or combinations described herein (e.g., a combination of histone acetylation code drugs (e.g., a BET inhibitor, promoter of HAT activity, MR antagonist, ALDH agonist, NRF2 activator, and/or HDAC inhibitor, etc.)) are delivered by parenteral administration (e.g., intramuscular, subcutaneous, intravenous, epidural, intracerebral, intracereroventricular, etc.). Formulations suitable for parenteral administration may include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants. Agents and combinations described herein (e.g., a combination of histone acetylation code drugs (e.g., a BET inhibitor, promoter of HAT activity, MR antagonist, ALDH agonist, NRF2 activator, and/or HDAC inhibitor, etc.)) may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally recognized in the field. For other parenteral injections, appropriate formulations may include aqueous or nonaqueous solutions, preferably with physiologically compatible buffers or excipients. Such excipients are generally recognized in the field.

In certain embodiments, delivery systems for pharmaceutical agents and combinations (e.g., a combination of histone acetylation code drugs (e.g., a BET inhibitor, promoter of HAT activity, MR antagonist, ALDH agonist, NRF2 activator, and/or HDAC inhibitor, etc.)) may be employed, such as, for example, liposomes and emulsions. In certain embodiments, compositions provided herein also include an mucoadhesive polymer, selected from among, for example, carboxymethylcellulose, carbomer (acrylic acid polymer), poly(methylmethacrylate), polyacrylamide, polycarbophil, acrylic acid/butyl acrylate copolymer, sodium alginate and dextran.

In some embodiments, an agent or combination of agents (e.g., a combination of histone acetylation code drugs (e.g., a BET inhibitor, promoter of HAT activity, MR antagonist, ALDH agonist, NRF2 activator, and/or HDAC inhibitor, etc.)) is administered in a therapeutically/prophylactically effective amount. Thus, a therapeutically/prophylactically effective amount is an amount that is capable of at least partially preventing or reversing a disease, disorder, or symptoms thereof. The dose required to obtain an effective amount may vary depending on the agent, formulation, disease or disorder, and individual to whom the agent is administered.

Determination of effective amounts may involve in vitro assays in which varying doses of agent are administered to cells in culture and the concentration of agent effective for ameliorating some or all symptoms is determined in order to calculate the concentration required in vivo. Effective amounts may also be based in in vivo animal studies.

Pharmaceutical compositions may be in unit dosage forms suitable for single administration of precise dosages. In unit dosage form, the formulation is divided into unit doses containing appropriate quantities of one or more agents therapeutically/prophylactically.

Dosing and administration regimes are tailored by the clinician, or others skilled in the pharmacological arts, based upon well-known pharmacological and therapeutic considerations including, but not limited to, the desired level of therapeutic effect, and the practical level of therapeutic effect obtainable.

In some embodiments, and upon the clinician's discretion, the administration of the compounds may be administered for an extended period of time, including throughout the duration of the patient's life in order to treat the disorder or ameliorate or otherwise control or limit the symptoms of the patient's disease.

In a case wherein the patient's status does improve, upon the clinician's discretion the administration of the agents agents (e.g., a combination of histone acetylation code drugs (e.g., a BET inhibitor, promoter of HAT activity, MR antagonist, ALDH agonist, NRF2 activator, and/or HDAC inhibitor, etc.)) may be given continuously; alternatively, the dose of drug being administered may be temporarily reduced or temporarily suspended for a certain length of time (i.e., a “drug holiday”). The length of the drug holiday can vary between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday may be from about 10% to about 100%, including, by way of example only, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In some embodiments, once improvement of the patient's symptoms/disorder/condition has occurred, a maintenance dose is administered if necessary. Subsequently, the dosage or the frequency of administration, or both, can be reduced, as a function of the symptoms, to a level at which the improved disease, disorder or condition is retained. Patients can, however, require intermittent treatment on a long-term basis upon any recurrence of symptoms.

In some embodiments, the amount of a given agent that will correspond to such an amount will vary depending upon factors such as the particular compound, disease and its severity, the identity (e.g., weight) of the subject or host in need of treatment, but can nevertheless be determined in a manner recognized in the field according to the particular circumstances surrounding the case, including, e.g., the specific agent being administered, the route of administration, the condition being treated, and the subject or host being treated. In general, however, doses employed for adult human treatment will typically be in the range of about 0.02-about 5000 mg per day, in some embodiments, about 1-about 1500 mg per day. The desired dose may conveniently be presented in a single dose or as divided doses administered simultaneously (or over a short period of time) or at appropriate intervals, for example as two, three, four or more sub-doses per day.

Provided in certain embodiments herein are combination therapies in which the agents and drug combinations provided herein (e.g., a combination of histone acetylation code drugs (e.g., a BET inhibitor, promoter of HAT activity, MR antagonist, ALDH agonist, NRF2 activator, and/or HDAC inhibitor, etc.)) are co-administered with one or more additional agents for the treatment of the disorder/condition, a side effect of the primary agent, or a comorbidity of the disorder/condition. Co-administered agents do not have to be administered in the same pharmaceutical composition, and may, because of different physical and chemical characteristics, have to be administered by different routes. Co-administered agents may be administered concurrently (in the same or separate formulations/compositions) or at separate times (separated by minutes, hours, days, etc.) The co-administered agents may be administered concurrently (e.g., simultaneously, essentially simultaneously or within the same treatment protocol) or sequentially, depending upon the nature of the disease, disorder, or condition, the condition of the patient, and the actual choice of agent used. The determination of the order of administration, and the number of repetitions of administration of each therapeutic agent during a treatment protocol, is well within the knowledge of the clinician after evaluation of the disease being treated and the condition of the patient.

Therapeutically-effective dosages can vary when the drugs are used in treatment combinations. Methods for experimentally determining therapeutically-effective dosages of drugs and other agents for use in combination treatment regimens are described in the literature. For example, the use of metronomic dosing, i.e., providing more frequent, lower doses in order to minimize toxic side effects, has been described extensively in the literature. Combination treatment further includes periodic treatments that start and stop at various times to assist with the clinical management of the patient.

For combination therapies described herein, dosages of the co-administered agents will of course vary depending on the type of co-drug employed, on the specific drug employed, on the disease being treated and so forth. In addition, when co-administered with one or more biologically active agents, the compound provided herein may be administered either simultaneously with the biologically active agent(s), or sequentially.

### REFERENCES

The following references, some of which are cited above by number, are herein incorporated by reference in their entireties.
1. Strahl B D & Allis C D (2000) The language of covalent histone modifications. Nature 403(6765):41-45.
2. Jenuwein T & Allis C D (2001) Translating the histone code. Science 293(5532):1074-1080.
3. Stillman B (2018) Histone Modifications: Insights into Their Influence on Gene Expression. Cell 175(1):6-9.
4. Baell J B, Leaver D J, Hermans S J, Kelly G L, Brennan M S, Downer N L, Nguyen N, Wichmann J, McRae H M, Yang Y, Cleary B, Lagiakos H R, Mieruszynski S, Pacini G, Vanyai H K, Bergamasco M I, May R E, Davey B K, Morgan K J, Sealey A J, Wang B, Zamudio N, Wilcox S, Garnham A L, Sheikh B N, Aubrey B J, Doggett K, Chung M C, de Silva M, Bentley J, Pilling P, Hattarki M, Dolezal O, Dennis M L, Falk H, Ren B, Charman S A, White K L, Rautela J, Newbold A, Hawkins E D, Johnstone R W, Huntington N D, Peat T S, Heath J K, Strasser A, Parker M W, Smyth G K, Street I P, Monahan B J, Voss A K, & Thomas T (2018) Inhibitors of histone acetyltransferases KAT6A/B induce senescence and arrest tumour growth. Nature 560(7717):253-257.
5. Haberland M, Montgomery R L, & Olson E N (2009) The many roles of histone deacetylases in development and physiology: implications for disease and therapy. Nat Rev Genet 10(1):32-42.
6. Lei I, Tian S, Gao W, Liu L, Guo Y, & Wang Z (2019) Acetyl-CoA production by specific metabolites promotes cardiac repair after myocardial infarction via mediating histone acetylation. bioRxiv Online Apr. 30, 2019.
7. Berry J M, Cao D J, Rothermel B A, & Hill J A (2008) Histone deacetylase inhibition in the treatment of heart disease. Expert Opin Drug Saf 7(1):53-67.
8. McKinsey T A (2012) Therapeutic potential for HDAC inhibitors in the heart. Annu Rev Pharmacol Toxicol 52:303-319.
9. Owen D J, Ornaghi P, Yang J C, Lowe N, Evans P R, Ballario P, Neuhaus D, Filetici P, & Travers A A (2000) The structural basis for the recognition of acetylated histone H4 by the bromodomain of histone acetyltransferase gcn5p. EMBO J 19(22):6141-6149.
10. Tamkun J W, Deuring R, Scott M P, Kissinger M, Pattatucci A M, Kaufman T C, & Kennison J A (1992) brahma: a regulator of Drosophila homeotic genes structurally related to the yeast transcriptional activator SNF2/SWI2. Cell 68(3):561-572.
11. Filippakopoulos P, Picaud S, Mangos M, Keates T, Lambert J P, Barsyte-Lovejoy D, Felletar I, Volkmer R, Muller S, Pawson T, Gingras A C, Arrowsmith C H, & Knapp S (2012) Histone recognition and large-scale structural analysis of the human bromodomain family. Cell 149(1):214-231.
12. Tian S, Lei I, Gao W, Liu L, Guo Y, Creech J, Herron T J, Xian S, Ma P X, Eugene Chen Y, Li Y, Alam H B, & Wang Z (2019) HDAC inhibitor valproic acid protects heart function through Foxm1 pathway after acute myocardial infarction. EBioMedicine 39:83-94.
13. Weeks K L (2019) HDAC inhibitors and cardioprotection: Homing in on a mechanism of action. EBioMedicine.
14. Lee H A, Lee D Y, Cho H M, Kim S Y, Iwasaki Y, & Kim I K (2013) Histone deacetylase inhibition attenuates transcriptional activity of mineralocorticoid receptor through its acetylation and prevents development of hypertension. Circulation research 112(7):1004-1012.
15. Olesen J B, Hansen P R, Abildstrom S Z, Andersson C, Weeke P, Schmiegelow M, Erdal J, Torp-Pedersen C, & Gislason G H (2011) Valproate attenuates the risk of myocardial infarction in patients with epilepsy: a nationwide cohort study. Pharmacoepidemiol Drug Saf 20(2): 146-153.
16. Hayes J D & Dinkova-Kostova A T (2017) Epigenetic Control of NRF2-Directed Cellular Antioxidant Status in Dictating Life-Death Decisions. Molecular cell 68(1):5-7.
17. Anand P, Brown J D, Lin C Y, Qi J, Zhang R, Artero P C, Alaiti M A, Bullard J, Alazem K, Margulies K B, Cappola T P, Lemieux M, Plutzky J, Bradner J E, & Haldar S M (2013) BET bromodomains mediate transcriptional pause release in heart failure. Cell 154(3):569-582.
18. Alqahtani A, Choucair K, Ashraf M, Hammouda D M, Alloghbi A, Khan T, Senzer N, & Nemunaitis J (2019) Bromodomain and extra-terminal motif inhibitors: a review of preclinical and clinical advances in cancer therapy. Future science OA 5(3):FSO372.

1. A system comprising a combination of therapeutic/prophylactic agents for administration to a subject for the treatment/prevention of organ/tissue injury, wherein the combination of therapeutic/prophylactic agents comprises two or more histone acetylation code agents.

2. The system of claim 1, wherein the two or more histone acetylation code agents are selected from a histone deacetylase (HDAC) inhibitor, a bromodomain and extraterminal-containing protein family (BET) inhibitor, a promoter of histone acetyl transferase (HAT) activity, a mineralocorticoid receptor (MR) antagonist, a nuclear factor erythroid 2-related factor 2 (NRF2) activator, and a aldehyde dehydrogenase (ALDH) agonist.

3. The system of claim 2, comprising an HDAC inhibitor.

4. The system of claim 3, wherein the HDAC inhibitor is selected from hydroxamic acid, depsipeptide, benzamide, electrophilic ketone, phenylbutyrate, valproic acid (VPA), a VPA derivative, and nicotinamide.

5. The system of claim 2, comprising a BET inhibitor.

6. The system of claim 5, wherein the BET inhibitor comprises a thienodiazepine moiety or a derivative or variant thereof.

7. The system of claim 5, wherein the BET inhibitor is selected from JQ1, I-BET 151, I-BET 762, OTX-015, TEN-010 (JQ2), CPI-203, CPI-0610, olinone, RVX-208, ABBV-744, LY294002, AZD5153, MT-1, and MS645.

8. The system of claim 2, comprising a MR antagonist.

9. The system of claim 8, wherein the MR antagonist is selected from spironolactone, eplerenone, canrenoic acid, canrenone, and drospirenone.

10. The system of claim 2, comprising a NRF2 activator.

11. The system of claim 10, wherein the NRF2 activator is selected from alpha-lipoic acid, curcumin, sulforaphane, resveratrol, polyresveratrol, genistein, andrographolidesiliphos, quercetin, dimethyl fumarate (DMF), oltipraz (4-methyl-5(pyrazinyl-2)-1-2-dithiole-3-thione), and ursodiol (ursodeoxycholic acid).

12. The system of claim 2, comprising a ALDH agonist.

13. The system of claim 12, wherein the ALDH agonist is selected from Alda-1, Alda-89, Alda-52, Alda-59, Alda-72, Alda-71, Alda-53, Alda-54, Alda-61, Alda-60, Alda-66, Alda-65, Alda-64, Alda-84

14. The system of claim 2, comprising a promoter of HAT activity.

15. The system of claim 14, wherein the promoter of HAT activity is selected from acetyl-CoA and a carbon source precursor to acetyl-CoA.

16. The system of claim 15, wherein the carbon source precursor to acetyl-CoA is selected from citrate, acetate, pyruvate, and octanoate.

17. The system of claim 1, wherein two or more histone acetylation code agents are combined into a single formulation.

18. The system of claim 1, wherein two or more histone acetylation code agents are separately formulated.

19. The system of claim 1, wherein two or more histone acetylation code agents are separately formulated but packaged together in a kit.

20. The system of claim 1, wherein two or more histone acetylation code agents are separately packaged.

21. A method of treating/preventing organ/tissue injury in a subject comprising co-administering two or more histone acetylation code agents to the subject.

22. The method of claim 21, wherein the two or more histone acetylation code agents are selected from a histone deacetylase (HDAC) inhibitor, a bromodomain and extraterminal-containing protein family (BET) inhibitor, a promoter of histone acetyl transferase (HAT) activity, a mineralocorticoid receptor (MR) antagonist, a nuclear factor erythroid 2-related factor 2 (NRF2) activator, and a aldehyde dehydrogenase (ALDH) agonist.

23. The method of claim 22, comprising an HDAC inhibitor.

24. The method of claim 23, wherein the HDAC inhibitor is selected from hydroxamic acid, depsipeptide, benzamide, electrophilic ketone, phenylbutyrate, valproic acid (VPA), a VPA derivative, and nicotinamide.

25. The method of claim 22, comprising a BET inhibitor.

26. The method of claim 25, wherein the BET inhibitor comprises a thienodiazepine moiety or a derivative or variant thereof.

27. The method of claim 25, wherein the BET inhibitor is selected from JQ1, I-BET 151, I-BET 762, OTX-015, TEN-010 (JQ2), CPI-203, CPI-0610, olinone, RVX-208, ABBV-744, LY294002, AZD5153, MT-1, and MS645.

28. The method of claim 22, comprising a MR antagonist.

29. The method of claim 28, wherein the MR antagonist is selected from spironolactone, eplerenone, canrenoic acid, canrenone, and drospirenone.

30. The method of claim 22, comprising a NRF2 activator.

31. The method of claim 30, wherein the NRF2 activator is selected from alpha-lipoic acid, curcumin, sulforaphane, resveratrol, polyresveratrol, genistein, andrographolidesiliphos, quercetin, dimethyl fumarate (DMF), oltipraz (4-methyl-5(pyrazinyl-2)-1-2-dithiole thione), and ursodiol (ursodeoxycholic acid).

32. The method of claim 22, comprising a ALDH agonist.

33. The method of claim 32, wherein the ALDH agonist is selected from Alda-1, Alda-89, Alda-52, Alda-59, Alda-72, Alda-71, Alda-53, Alda-54, Alda-61, Alda-60, Alda-66, Alda-65, Alda-64, Alda-84

34. The method of claim 22, comprising a promoter of HAT activity.

35. The method of claim 34, wherein the promoter of HAT activity is selected from acetyl-CoA and a carbon source precursor to acetyl-CoA.

36. The method of claim 35, wherein the carbon source precursor to acetyl-CoA is selected from citrate, acetate, pyruvate, and octanoate.

37. The method of claim 21, wherein two or more histone acetylation code agents are combined into a single formulation.

38. The method of claim 21, wherein two or more histone acetylation code agents are separately formulated.

39. The method of claim 21, wherein two or more histone acetylation code agents are separately formulated but packaged together in a kit.

40. The method of claim 21, wherein two or more histone acetylation code agents are separately packaged.

41. The method of claim 21, wherein the subject has suffered a tissue and/or organ injury.

42. The method of claim 21, wherein the subject suffers from a disease or condition, or has suffered a physiological event, that causes tissue and/or organ injury.

43. The method of claim 21, wherein the subject is at elevated risk of a disease, condition, or physiological event, that causes tissue and/or organ injury.

44. The method of one of claims 41-43, wherein the tissue and/or organ injury comprises cardiac damage.

45. The method of one of claims 41-43, wherein the tissue and/or organ injury comprises ischemic damage.

46. The method of claim 44, where the subject has suffered a myocardial infarction.

47. The method of claim 21, wherein the histone acetylation code agents are administered orally or parenterally.

48. A method of treating/preventing organ damage comprising (a) intravenously administering in clinical setting a first combination of histone acetylation code agents to a subject that has suffered and ischemic event; and (b) orally administering a second combination of histone acetylation code agents to the subject.

49. The method of claim 48, wherein the first combination and the second combination comprise the same histone acetylation code agents, but are formulated for intravenous and oral administration, respectively.

50. The method of claim 48, wherein the first combination and the second combination comprise different histone acetylation code agents.
